(19) United States
(12) Patent Application Publication
(10) Pub. No.: US 2014/0194400 A1
(43) Pub. Date: Jul. 10, 2014

(54) Nasal Pharmaceutical Formulation

(71) Applicants: Annegret Hildebrand-Cyrener, Radebeul (DE); Joachim Maus, Muehlheim am Main (DE); Ullrich Munzel, Woellstadt (DE); Hans Tritschler, Muenchen (DE); Mario Weingart, Dresden (DE)

(72) Inventors: Annegret Hildebrand-Cyrener, Radebeul (DE); Joachim Maus, Muehlheim am Main (DE); Ullrich Munzel, Woellstadt (DE); Hans Tritschler, Muenchen (DE); Mario Weingart, Dresden (DE)

(73) Assignee: Meda Pharma GmbH & Co., Kg, Bad Homburg (DE)

(21) Appl. No.: 14/122,561

(22) PCT Filed: May 24, 2012
(86) PCT No.: PCT/EP2012/002222

§ 371 Date: Feb. 19, 2014

(30) Foreign Application Priority Data
May 27, 2011 (DE) 10 2011 103 347.9

(51) Int. Cl. A61K 9/00; A61K 31/56

(52) U.S. Cl. CPC A61K 9/0043; A61K 31/56
USPC 514/180

(57) ABSTRACT

The present invention relates to a nasel formulation comprising as its active ingredient an intranasal corticosteroid, and also to a method for prophylaxis or treatment of seasonal or perennial allergic and non-allergic rhinitis and rhinoconjunctivitis.

The present invention relates to a nasal formulation comprising an intranasal corticosteroid as active ingredient. In a preferred embodiment, the invention relates to a nasal formulation comprising fluticasone or pharmaceutically acceptable esters or salts thereof. In a particularly preferred embodiment, the invention relates to a nasal formulation comprising fluticasone propionate.

The present invention further relates to a method for the prophylaxis or treatment of seasonal or perennial allergic and non-allergic rhinitis and rhinoconjunctivitis and also for the treatment of nasal polyps, for prophylaxis of polyp recurrence following surgical removal of nasal polyps, as adjuvant therapy for acute and chronic sinusitis, for sleep apnea, snoring or inflammation-related obstructive sleep disorders, with a nasal formulation comprising an intranasal corticosteroid as active ingredient, preferably fluticasone or pharmaceutically acceptable esters or salts thereof. In a particularly preferred embodiment, the invention relates to a method for the prophylaxis or treatment of seasonal or perennial allergic rhinitis and rhinoconjunctivitis with a nasal formulation comprising fluticasone propionate.

The present invention further relates to a method for preparing a nasal formulation comprising an intranasal corticosteroid as active ingredient, preferably fluticasone or pharmaceutically acceptable esters or salts thereof. In a preferred embodiment, the invention relates to a method for preparing a nasal formulation comprising fluticasone propionate.

Allergic rhinitis is a global health problem with increasing prevalence. Currently about 500 million people worldwide are affected by it. Symptoms of allergic rhinitis affect social life, sleep, the ability to learn and work and therefore cause considerable stress (Bousquet et al., Allergy. 2008 April; 63 Suppl 86:8-160).

For patients with stronger symptoms, particularly nasal congestion, intranasal corticosteroids are the treatment of choice (LaForce J Allergy Clin Immunol 1999; 103; pp. 388-94; Brozek et al., J Allergy Clin Immunol 2010; 126: 466-76, Wallace J Allergy Clin Immunol. 2008 August; 122 (2 Suppl): pp. 1-84).

Fluticasone is an active ingredient from the corticosteroid class and is used for the treatment of seasonal or perennial allergic rhinitis. Formulations on the market for nasal application are, for example, Flutide, Flonase or Fluticasone Propionate Nasal Spray 50 μg (Roxane Laboratories). In the suspensions, the active ingredient fluticasone is present as a microfine dispersion in the liquid.

Research shows, however, that more than 60% of patients with allergic rhinitis are not satisfied with their current treatment, particularly due to lack of efficacy (Bousquet, J Allergy Clin Immunol. 2009 September; 124(3): 428-33). Thus, there exists a need for improved medicaments for the treatment of allergic rhinitis.

The object of the present invention is to provide a corticosteroid-containing medicament for the treatment of allergic rhinitis with improved efficacy. The object is achieved by means of a nasal formulation of fluticasone, particularly fluticasone propionate, comprising microcrystalline cellulose+Na carboxymethylcellulose (Avicel CL 611), disodium edetate, polysorbate 80, glycerine, benzalkonium chloride and phenylethyl alcohol as auxiliaries. The nominal dose of fluticasone propionate is 50 μg.

A critical parameter for the efficiency of locally applied and locally acting substances is the nominal dose of active ingredient administered. It is generally assumed that drugs with the same nominal dose of the same active ingredient show comparable effects (LeSouef, Allergy 1999, 54, pp. 93-96).

The formulation according to the invention has the advantage, compared to the prior art, that the corticoid fluticasone has a better local availability in the nose despite the same nominal dose (Derendorf et al., 2012 Br J Clin Pharmacol accepted) and can have a stronger effect there.

Table 1 shows a comparison between the inventive formulation according to example 1 and a formulation from the prior art (Fluticasone Propionate Nasal Spray 50 μg (Roxane Laboratories)) using the same nominal dose. The results are reported as the difference from baseline unless indicated otherwise (rTNSS: reflective Total Nasal Symptom Score; iTNSS: instantaneous Total Nasal Symptom Score; TOSS: Total Ocular Symptom Score).

TABLE 1

| | | Comparison |
|---|---|---|
| | | (Fluticasone |
| | Inventive | 50 μg from |
| Parameter | formulation | Roxane) |
| rTNSS | −5.1 | −3.8 |
| iTNSS | −4.60 | −3.46 |
| rTOSS | −2.71 | −2.17 |
| Nasal congestion | −1.10 | −0.86 |
| Nasal itching | −1.10 | −0.91 |
| Ocular itching | −0.96 | −0.70 |
| Watery eyes | −0.96 | −0.82 |
| rTNSS (baseline | −5.42 | −4.76 |
| >18.9) | | |
| rTNSS (blocker) | −4.95 | −3.92 |
| Nasal congestion | −1.26 | −0.90 |
| (blocker) | | |

Compared to before treatment, the nasal and ocular symptom scores and also the individual complaints decrease more distinctly than with conventional fluticasone nasal spray at the same nominal dose. While conventional fluticasone alters the overall score of the four relevant nasal symptoms (nasal congestion, sneezing, runny nose, nasal itching) on average by only 3.8 points on a scale of 0 to 24 during 14-day therapy (Hampel et al., Ann Allergy Asthma Immunol. 2010; 105: pp. 168-73), the new formulation performs distinctly better at 5.1 points (Carr et al., J Allergy Clin Immunol 129(5) 2012 pp. 1282-1289).

As mentioned above, the superior efficacy depends on the better local availability of the active ingredient which is reflected in the better systemic bioavailability. The systemically available fluticasone must have been mainly absorbed through the nasal mucosa, since oral absorption is only about 1%. The improved bioavailability has been demonstrated in the study of Derendorf et al., 2012.

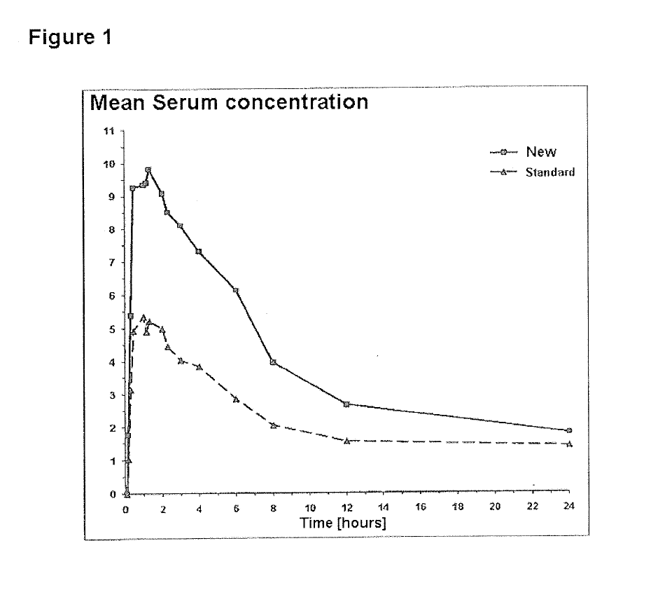

In one of two randomized, 3-period, 6-sequence, 3-treatment crossover studies, 19 healthy volunteers were each once intranasally administered 200 μg of fluticasone (nominally 50 μg, 2 sprays in each nostril) as conventional standard (Fluticasone Propionate Nasal Spray 50 μg (Roxane Laboratories)) and in the inventive formulation (new) according to example 1. Serum fluticasone was measured over 24 hours. The mean fluticasone levels in [pg/ml] are plotted against time in FIG. 1 and show the degree of improvement in the availability.

Further embodiments of the invention comprise, in place of fluticasone or a pharmaceutically acceptable ester or salt thereof, one or more active ingredients from the group of intranasal corticosteroids consisting of budesonide, beclomethasone, mometasone, triamcinolone, dexamethasone, ciclesonide or pharmaceutically acceptable salts or esters thereof.

The formulation optionally comprises one or more auxiliaries from the group of suspension agents/thickeners, such as carboxymethylcellulose, hydroxymethylcellulose, methylcellulose, gelatine, polyvinylpyrrolidone, polyethylene glycol, polyvinyl alcohol, preferably microcrystalline cellulose+Na carboxymethylcellulose (Avicel CL 611), chelating agents, preferably disodium edetate, wetting agents such as polyoxyethylene derivatives of fatty acids or polyoxyethylene derivatives of partial fatty acid esters of sorbitol anhydrides, preferably polysorbate 80, osmotically active substances such as sucrose, glucose, sorbitol, propylene glycol, NaCl, preferably glycerol, and also preservatives such as thiomersal, benzyl alcohol, alkonium and benzalkonium salts, chlorhexidine gluconate, preferably benzalkonium chloride and phenylethyl alcohol.

The preparation of the formulation according to the invention is carried out, for example, by heating purified water to 30-40° C. Disodium edetate and glycerol are then successively added and both are mixed for ca. 5 min. Microcrystalline cellulose and Na carboxymethylcellulose are sieved through a 40 mesh sieve and are then added with stirring and the mixture is further stirred for ca. 30 min.

In a separate vessel, polysorbate 80 is stirred with purified water for ca. 5 min. Fluticasone propionate is added with further stirring and the mixture is further stirred for ca. 30 min.

The two dispersions are combined and are further mixed for ca. 10 min. Benzalkonium chloride solution 10% (w/v) is added and the mixture is mixed by stirring for ca. 10 min.

Phenylethyl alcohol is added and the mixture is mixed by stirring for ca. 10 min. After addition of purified water, the suspension is homogenized for ca. 30 min. and is passed through a 200 mesh sieve.

The administration of the formulation is effected by spray bottles with commercially available pumps, such as those from Aptar or MeadWestvaco Corporation. The VP3/140F CS20-AG pump from Aptar is particularly preferred.

The formulation according to the invention is applied with a droplet size of no more than 150 μm, preferably between 50 μm and 100 μm, particularly preferably between 75 μm and 95 μm, in half of the droplets in the administered dose unit.

One dose unit comprises between 10 and 200 μg, preferably between 25 and 100 μg, particularly preferably between 40 and 60 μg of intranasal corticosteroid. One dose unit comprises, for example, 50 μg of fluticasone propionate.

The dose unit of the intranasal corticosteroid is administered in a volume between 50 and 250 μl, preferably between 100 and 150 μl. A dose unit of fluticasone propionate is administered, for example, in a volume of 137 μl per spray.

1-2 sprays per nostril are administered once or twice daily and therefore in total 2-8 sprays per day; particular preference is given to administering 1 spray in the morning and 1 spray in the evening per nostril and therefore in total 4 sprays per day.

### EXAMPLES

The following compositions are listed by way of example without restricting the invention.

### Example 1

| | Amount |
|---|---|
| Ingredient | [g/100 g] |
| Fluticasone | 0.0365 |
| propionate | |
| MCC + NaCMC** | 2.00 |
| (Avicel CL | |
| 611) | |
| Disodium | 0.01 |
| edetate | |
| Polysorbate 80 | 0.005 |
| Glycerol | 2.30 |
| Benzalkonium | 0.01 |
| chloride | |
| Phenylethyl | 0.25 |
| alcohol | |
| Purified water | to 100 |

### Example 2

| | Amount |
|---|---|
| Ingredient | [g/100 g] |
| Fluticasone | 0.025 |
| propionate | |
| MCC + NaCMC** | 2.00 |
| (Avicel CL | |
| 611) | |
| Disodium | 0.01 |
| edetate | |
| Polysorbate 80 | 0.005 |
| Glycerol | 2.30 |
| Benzalkonium | 0.01 |
| chloride | |
| Phenylethyl | 0.25 |
| alcohol | |
| Purified water | to 100 |

1. A nasal pharmaceutical formulation comprising fluticasone or a pharmaceutically acceptable ester or salt thereof and optionally one or more auxiliaries.

2. The formulation as claimed in claim 1, characterized in that fluticasone propionate is used.

3. The formulation as claimed in claim 1, wherein one or more auxiliaries are present from the group consisting of suspension agents, chelating agents, wetting agents, osmotically active substances and preservatives.

4. The formulation as claimed in claim 3, wherein the suspension agent present is microcrystalline cellulose and Na carboxy-methylcellulose (Avicel CL 611).

5. The formulation as claimed in claim 3, wherein the chelating agent present is disodium edetate.

6. The formulation as claimed in claim 3, wherein the wetting agent present is polysorbate 80.

7. The formulation as claimed in claim 3, wherein the osmotically active substance present is glycerol.

8. The formulation as claimed in claim 3, wherein at least one preservative is present from the group comprising benzalkonium chloride and phenylethyl alcohol.

9. The formulation as claimed in claim 1, wherein the formulation is administered by means of a spray pump.

10. A method for the prophylaxis or treatment of allergies seasonal or perennial rhinitis, comprising administering a pharmaceutically effective amount of the formulation as claimed in claim 1, for the prophylaxis or treatment of allergic seasonal or perennial rhinitis or rhinoconjunctivitis.
